(19) United States
(12) Patent Application Publication
(10) Pub. No.: US 2022/0062350 A1
(43) Pub. Date: Mar. 3, 2022

(54) OIL-IN-WATER EMULSION GUMMY COMPOSITION

(71) Applicant: PHARMAVITE LLC, West Hills, CA (US)

(72) Inventors: Haiyan Ge, Valencia, CA (US); Javier Pacheco, Sylmar, CA (US)

(73) Assignee: PHARMAVITE LLC, West Hills, CA (US)

(21) Appl. No.: 17/012,009

(22) Filed: Sep. 3, 2020

(51) Int. Cl. A61K 35/60; A61K 31/12; A61K 47/24; A61K 31/355; A61K 47/36; A61K 47/02; A61K 47/42; A61K 47/10

(52) U.S. Cl. CPC A61K 35/60; A61K 31/12; A61K 47/24; A61K 47/10; A61K 47/36; A61K 47/02; A61K 47/42; A61K 31/355

(57) ABSTRACT

A gummy composition including a set emulsification of an oil phase in a water phase, wherein the water phase comprises a structurant, and wherein the gummy composition has a hardness of 150 gForce or greater and a water activity below 0.73. A method including administering a gummy composition including a set emulsification of an oil phase in a water phase and the gummy composition includes a hardness of 150 gForce or greater and a water activity below 0.73.

### FIELD

Nutritional compositions particularly gummy compositions and methods related thereto.

### BACKGROUND

Chewable gummy (gum) products or compositions generally made of gelatin or pectin matrix with sugar, glucose, corn syrup, flavoring, coloring and citric acid have been a popular snack food product. The product (composition) typically has a gel or gel-like structure and texture with a length on the order of two centimeters (cm) and is produced in a variety of shapes, colors and flavors that are chewable when consumed. Recently, gummy products have been supplemented with vitamins, minerals, essential oils and other nutritional supplements to provide a nutritional supplement that appeals to children and adults that do not like to swallow or have difficulty swallowing tablets or capsules.

Gummy compositions are often formed as a water-based gummy slurry of a gelatin and sugar mixture. The gummy slurry is mixed at an elevated temperature (e.g., 70° C. to 100° C.) to produce a flowable liquid. The flowable liquid is poured into a mold and allowed to set. A conventional mold is a corn starch mold. A corn starch mold is formed by stamping a desired gummy shape on a tray filled with corn starch powder. Once in the mold, the gummy composition is cooled and allowed to set. After the gummy composition has set, i.e. completely solidified, the tray is tipped over, breaking the mold and separating the gelled gummy composition from the corn starch. The starch mold generally functions to reduce a temperature of the gummy composition and to absorb water from the gummy. Both help with the gummy solidification. Usually it takes about 24 hours for the gummy composition to completely set with a “set time” defined as the time it takes for a gummy composition to form a firm gelled structure throughout—from an outer surface to a midpoint when measured on each side or face. Once the gummy composition has set, the gummy composition may be destarched and coated (for example with carnauba wax).

Starchless production of gummy compositions offers advantages such as good hygiene and generally fast setting (gelation) over conventional production. An example of a starchless mold is a silicone mold.

### BRIEF DESCRIPTION OF THE DRAWINGS

FIG. 1 shows a three-dimensional model of a fish oil oil-in-water gummy composition formulation probability of setting when a structurant (gelatin) weight percent is 5.87 percent;

FIG. 2 shows a three-dimensional model of a fish oil oil-in-water gummy composition formulation probability of setting when a structurant (gelatin) weight percent is 7.52 percent;

FIG. 3 shows a graph of compression force versus time for two compressions into an emulsion gummy composition including fish oil and Vitamin E using a TA-25 cylindrical probe;

FIG. 4 shows a three-dimensional model of a magnesium and calcium oil-in-water emulsion gummy composition formulation probability of setting when a weight percent of an oil (medium chain triglycerides) is 21.56 percent;

FIG. 5 shows a three-dimensional model of a magnesium and calcium oil-in-water emulsion gummy composition formulation probability of setting when a weight percent of an oil (medium chain triglycerides) is 26 percent;

FIG. 6 shows three-dimensional plots of probability of setting of an oil-in-water emulsion gummy composition containing minerals (calcium and magnesium) and structurants of gelatin, locust bean gum and agar-agar with 1) and 2) : either an amount of locust bean gum held constant and varying an amount of gelatin, water and agar-agar or 3) and 4): an amount of gelatin held constant and varying an amount of locust bean gum, water and agar-agar;

FIG. 7 shows a three-dimensional plot of hardness of an oil-in-water emulsion gummy composition containing minerals (calcium and magnesium) and structurants of gelatin, locust bean gum and agar-agar with a locust bean gum amount maintained at 0.294 weight percent and an amount of water, agar-agar and gelatin varied;

FIG. 8 shows a graph of compression force versus time for two compressions into an oil-in-water emulsion gummy composition including magnesium and calcium using a TA-25 cylindrical probe; and

FIG. 9 shows two, three-dimensional plots of chewiness of an oil-in-water emulsion gummy composition containing minerals (calcium and magnesium) and structurants of gelatin, locust bean gum and agar-agar with an amount of water held constant in a composition and an amount of each structurant changed.

### DETAILED DESCRIPTION

A chewable gummy or gum composition for oral consumption in a set emulsification of an oil phase in a water phase is disclosed. The water phase may include a structurant, a sugar alcohol such as maltitol syrup, sugar and water. The structurant may be, for example, gelatin, pectin, agar (or agar-agar), locust bean gum or a combination of one or more structurants. By selecting an amount (e.g., weight percent) of the components of the composition, primarily of the water phase components, a gummy composition may be provided that possesses a relatively rapid removal time and corresponding rapid set time, good texture, good heat stability and a pleasant taste. In terms of measurable physical properties, a suitable oil-in-water set emulsion gummy composition has a hardness of 150 gForce or greater measured as a peak force using a TA-8A probe to push through a gummy composition sample from a top surface for 5 millimeters (mm) at a speed of 0.5 millimeters per second (mm/sec.), the gummy composition sample having a square shape of 2.0 centimeters (cm) by 2.0 cm with thickness of 0.9 cm; a water activity below 0.73; a ° Brix of 77 to 83; and a removal time of 30 minutes or less while providing a texture and appearance acceptable to consumers. A “removal time,” “removable time” or “release time” as used herein is a time that it takes for at least an outer portion of a gummy composition to gel so that the gummy composition may be removed from a starchless mold as a single unit. A “set time” as used herein is defined as the time it takes for a gummy composition to form a firm gelled structure throughout-from an outer surface to a midpoint when measured on each side or face. A set time for a gummy composition is generally longer than a removal time. For gummy compositions, a removal time is directly related to a set time. Therefore, a reduction in a removal time will result in a reduction in a set time and an increase in the efficiency of a gummy composition manufacture process. An oil phase of a gummy or gum composition may contain a lecithin, a polyunsaturated fatty acid, e.g. an omega-3 fatty acid, an omega-6 fatty acid, an omega-9 fatty acid or a vegetable oil, such as one or a mixture of fatty acid esters (for example phospholipids, mono-, di- or tri-glycerides, and lower alkyl esters). Such materials may be natural, synthetic or semi-synthetic. The use of plant and marine oils (e.g. oils from plant seeds (e.g., sunflower oil or soybean oil), algae, fish (especially oily fish), microorganisms and marine invertebrates (e.g., krill)) is especially preferred as is the use of docosahexaenoic acid (DHA) and/or eicosapentaenoic acid (EPA) triglycerides or ethyl esters. An oil phase of a gummy or gum composition may also include medium chain triglyceride (MCT) oils such as coconut or palm kernel oil, soluble vitamin oil such as Vitamin A, D, E, K12, olive oil and/or avocado oil.

To form a gummy composition, the water phase and oil phase are separately prepared. For the water phase, the structurant(s) may be hydrated followed by the addition of other components (e.g., sugar alcohol, sugar). The oil phase is then mixed with the water phase at a mixing speed sufficient to form an emulsification of micrometer or less size droplets of the oil phase uniformly dispersed throughout the water phase. After emulsifying, the mixture is poured into molds of a desired shape and size for an edible gummy composition and allowed to at least begin to set. By selecting the appropriate amount of components in the gummy composition, a removal time of 30 minutes or less can be achieved that provides a hardness of 150 gForce or greater measured as a peak force using a TA-8A probe to push through the sample (a square shape of 2.0 centimeters (cm) by 2.0 cm with thickness of 0.9 cm) from a top surface for 5 mm at a speed of 0.5 mm/sec., good texture and good heat stability.

I. Fish Oil Emulsion Gummy Compositions

Fish oil emulsion gummy compositions can incorporate a relatively high amount of fish oil into gummy by dispersing micro-size fish oil droplets into water phase uniformly. In one example, a relatively high amount is at least 10 percent of the gummy composition by weight, or between 10 and 45 percent, or between 10 and 40 percent, or between 10 and 35 percent or between 10 and 30 percent, or between 10 and 25 percent or between 10 and 20 percent.

The fish oil emulsion gummy composition is an oil-in-water emulsion. The water phase may be described as the continuous phase, that contains a structurant or structurants (e.g., gelatin, pectin, agar-agar, locust bean gum), water, sugar or/and xylitol (solid), maltitol (liquid) and citric acid for sour taste. A suitable structurant of a gelatin may be a gelatin having a bloom of 150 or greater, such as 150 bloom to 300 bloom, such as 175 bloom to 270 bloom or such as 175 bloom to 250 bloom. The oil phase contains the fish oil only, or fish oil with a carrier oil such as medium chain triglyceride (MCT) oil, sunflower oil, soybean oil, etc. Previous lab formula batches show that triglyceride (TG) type fish oil or ethyl ester (EE) type fish oil can form emulsion gummy compositions. However, when the weight percentage of ingredients in the water phase changes, sometimes desirable firm gummy compositions did not form. Therefore, a mixture Design of Experiment (DOE 1) was designed to understand the effect of the interaction of ingredients in the water phase on gummy setting, gummy hardness, water activity, ° Brix, pH, and heat stability.

A. Design of Experiment 1

In DOE 1, the oil phase of the gummy composition consisted of fish oil (triglyceride), masking flavor, lemon flavor, and, optionally, silica. Each of the ingredients or components of the oil phase except the silica are kept the same weight percentage in the formula: fish oil 10.03%, masking flavor 0.5% and lemon flavor 1.22%, listed as ingredients #7-#9 in Table 2. The water phase utilized up to six ingredients or components that were treated as variables. The weight percent ranges for the variables of silica (in oil phase) and the ingredients of the water phase are provided in Table 1.

TABLE 1
Variable Ingredients in DOE 1, all in the
water phase except silica (oil phase)

| | Minimum | Maximum |
|---|---|---|
| Mixture DOE | (wt. %) | (wt. %) |
| Water | 15.00 | 25.00 |
| 250 Bloom gelatin | 5.00 | 8.00 |
| Maltitol syrup | 24.00 | 36.00 |
| Sugar | 0.00 | 40.00 |
| Xylitol | 0.00 | 40.00 |
| Silica | 0.00 | 0.50 |
| 50% Citric acid solution | 0.00 | 1.00 |

| | |
|---|---|
| Total: | 88.25 |

33 DOE batches were designed and conducted, and gummy set time, hardness, water activity, Brix, pH and heat stability were tested. Table 2 shows two representative formulations of the DOE 1 batches that can form emulsion gummy compositions and the physical properties of samples made according to the two formulations.

For DOE 1 Batch #A1, the gelatin is initially hydrated in water with a Caframo overhead mixer at about 90° C. for 15 minutes and then maltitol, sugar and xylitol were added while mixing to be dissolved. The ingredients in the oil phase are ingredients #7-#9 in Table 2. The ingredients in the oil phase were mixed together first before being added into the water phase at 70° C. with a Silverson L5M-A high shear mixer at the mixing speed 3000 revolutions per minute (rpm) to 6000 rpm. The final samples were then deposited into the silicone molds.

For DOE 1 Batch #A2, the water phase batch procedure is the same as Batch #A1. The oil phase, however, contains 0.5 wt % hydrophobic fumed silica (Ingredient #6, Aerosil R972 Pharma) that was added into the oil phase with Silverson L5M-A high shear mixer first and then the oil phase was added into the water phase at 70° C. at the mixing speed about 6000 rpm.

After mixing the oil and water phases, the gummy composition samples were deposited into silicone molds to set. A removal time was established by pushing gummy composition samples out from the back of a silicone mold and confirming that all sides of the samples were separated from the mold. After the gummy composition samples were removed from the mold, the samples were allowed to sit in room air for 24 hours until the following physical property tests were performed: Brix (Brix Refractometer), water activity (Aqualab 4TE water activity meter) and hardness (Texture Analyzer from Stable Micro Systems). For a hardness measurement, a TA-8A probe was used to push through the sample (a square shape of 2.0 centimeters by 2.0 centimeters with thickness of 0.9 centimeters) from a top surface for 5 mm at a speed of 0.5 mm/sec. The peak force was measured as the hardness. Five samples were tested, and the average hardness was used. For the heat stability study, samples were packed into bottles and put into a 40° C./75 percent relative humidity (RH %) stability chamber. The sample appearance after seven days was evaluated after the samples were removed from the chamber and returned to the room temperature.

TABLE 2
DOE 1 Batch Formulations and their
Physical Properties

| | | Batch #A1 | Batch #A2 |
|---|---|---|---|
| | Ingredients | wt % | wt % |
| 1 | Water | 18.34% | 18.10% |
| 2 | 250 Bloom Gelatin Porcine | 8.00% | 5.95% |
| | (Gelita) | | |
| 3 | Maltidex Maltitol Syr 5275 | 24.00% | 24.00% |
| | (Cargill) | | |
| 4 | Sugar (Prinova USA) | 18.11% | 33.62% |
| 5 | Xylitol (Danisco) | 19.80% | 6.08% |
| 6 | Aerosil R972 Pharma | 0.00% | 0.50% |
| | (Evonic) | | |
| 7 | Fish Oil TG1055 (Golden | 10.03% | 10.03% |
| | Omega) | | |
| 8 | Masking flavor QN94 | 0.50% | 0.50% |
| | (Virginia Dare) | | |
| 9 | Lemon flavor (Virginia Dare) | 1.22% | 1.22% |
| | Total: | 100.00% | 100.00% |
| | Removal time (minutes) | 10 | 40 |
| | ° Brix (%) | 82.0 | 81.5 |
| | Water Activity | 0.6438 | 0.6735 |
| | TA-8A Hardness (g * Force) | 154 | 118 |
| | Appearance after 7 days in | Shape not | Shape |
| | 40° C./75%RH chamber | changed | slightly |
| | | | deformed |
| | | | but not |
| | | | melt |

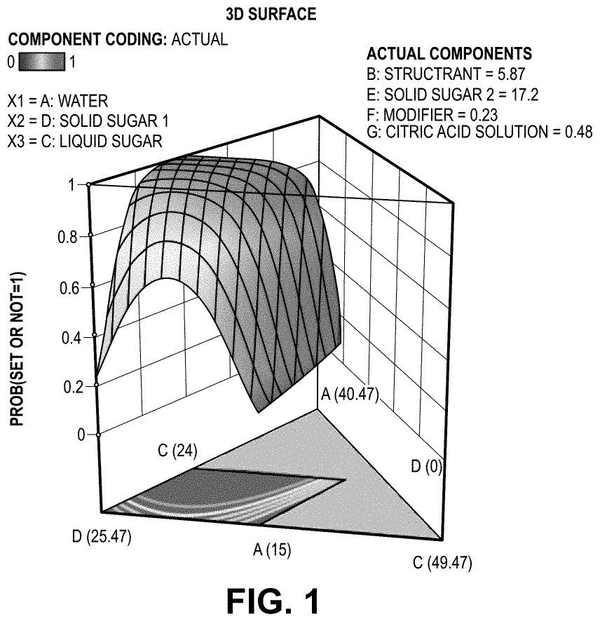

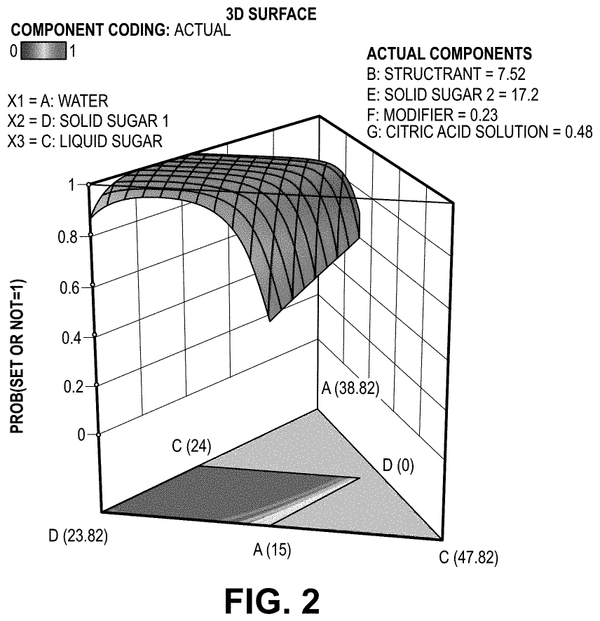

Whether a gummy or gum composition adequately sets or not depends on the ingredients or components identified as variables. DOE modeling showed that water, structurant, and the interaction of maltitol syrup and sugar have the most positive impact on the setting, while silica (in oil phase) and maltitol syrup have the most negative impact on the setting. FIG. 1 and FIG. 2, for example, show that increasing a weight percent of a structurant (gelatin) in the water phase from 5.87 weight percent (wt %) to 7.52 wt %, greatly increases the probability of a gummy formulation that will set or set within a desired time frame. Whether a gummy composition sets was evaluated by cutting gummy compositions into halves and checking whether a firm structure was formed in the center.

Whether a gummy or gum composition has adequate hardness also depends on the ingredients or components identified as variables. DOE modeling showed that the structurant and silica tend to increase the hardness the most, followed by maltitol syrup, water etc. The interaction of maltitol syrup and xylitol tend to reduce the hardness.

Formulations for targeted physical property ranges were created using the DOE 1 analysis-built models for each physical property linked to the various ingredients. Table 3 presents a targeted formulation range based on the DOE 1 analysis. The targeted formulation will provide properties of ° Brix range between 77 and 83, such as 78-81; water activity less than 0.73, such as less than 0.70; removal time of 10 minutes to 30 minutes; and hardness greater than 150 gForce, such as 150 gForce to 600 gForce, using a TA-8A probe to push through the sample (a square shape of 2.0 centimeters by 2.0 centimeters with thickness of 0.9 centimeters) from the top surface for 5 millimeters (mm) at a speed of 0.5 mm/sec. A gummy composition formed according to the targeted formulation range will have good heat stability at 40° C./75% RH for at least seven days as well as good firmness and chewiness.

TABLE 3
Targeted Formulation Ranges for Fish
Oil Gummy Composition

| | | wt % | Example |
|---|---|---|---|
| | Ingredients | range | wt % |
| 1 | Water | 16.0-23.0 | 17.50 |
| 2 | 250 Bloom Gelatin Porcine | 5.00-8.50 | 7.76 |
| 3 | Maltitol Syrup | 16.0-35.0 | 24.78 |
| 4 | Sugar | 15.0-26.0 | 19.00 |
| 5 | Xylitol | 8.0-21.0 | 13.63 |
| 6 | Hydrophobically-modified | 0-0.50 | 0.00 |
| | silica | | |
| 7 | Fish Oil | 5.0-45.0 | 14.83 |
| 8 | Masking flavor | 0.2-1.0 | 0.50 |
| 9 | Vitamin E tocopherol | 0.2-0.8 | 0.45 |
| 10 | Flavor | 0.2-1.0 | 0.50 |
| 11 | Citric acid solution 50% | 0-1.2 | 0.80 |
| 12 | Color | 0.05-0.50 | 0.25 |
| | | | 100.00 |

Table 4 shows two examples of gummy composition formulations made according to the target formulation of Table 3. The physical properties of gummy composition samples made using the two formulations are shown at the bottom of the table.

TABLE 4
Targeted DOE 1 Formulations and their
Physical Properties

| | | Batch #B1 | Batch #B2 |
|---|---|---|---|
| | Ingredients | wt % | wt % |
| 1 | Water | 22.04% | 22.91% |
| 2 | 250 Bloom Gelatin Porcine | 7.34% | 8.00% |
| | (Gelita) | | |
| 3 | Maltidex Maltitol Syr 5275 | 24.00% | 24.95% |
| | (Cargill) | | |
| 4 | Sugar (Prinova USA) | 25.78% | 18.94% |
| 5 | Xylitol (Danisco) | 8.95% | 13.33% |
| 6 | Aerosil R972 Pharma | 0.14% | 0.00% |
| | (Evonic) | | |
| 7 | Fish Oil TG1055 (Golden | 10.03% | 10.03% |
| | Omega) | | |
| 8 | Masking flavor QN94 | 0.50% | 0.50% |
| | (Virginia Dare) | | |
| 9 | Lemon flavor (Virginia | 1.22% | 1.22% |
| | Dare) | | |
| 10 | Citric acid solution 50% | 0.00% | 0.12% |
| | Total: | 100.00% | 100.00% |
| | Removal time (minutes) | 12 | 10 |
| | Brix (%) | 79.0 | 78.0 |
| | Water Activity | 0.7172 | 0.7198 |
| | TA-8A Hardness (g * Force) | 180 | 200 |
| | Appearance after 7 days in | Shape not | Shape not |
| | 40° C./75%RH chamber | changed | changed |

Soon after the gummy composition samples were deposited, the removal time presented in Table 4 was established by pushing the samples out from the back of the silicone mold in which they were deposited and confirming that all sides of the samples were separated from the mold. After the gummy composition samples were removed from the mold, the samples were allowed to sit in room air for 24 hours until the following physical property tests were performed: Brix (Brix Refractometer), water activity (Aqualab 4TE water activity meter) and hardness (Texture Analyzer from Stable Micro Systems). For a hardness measurement, a TA-8A probe was used to push through the sample (a square shape of 2.0 centimeters by 2.0 centimeters with thickness of 0.9 centimeters) from the square top surface for 5 millimeters (mm) at a speed of 0.5 mm/sec. The peak force was measured as the hardness. Five samples were tested, and the average hardness was used. For the heat stability study, samples were packed into bottles and put into a 40° C./75 percent relative humidity (RH %) stability chamber. The sample appearance after seven days was evaluated after the samples were pulled out of the chamber and returned to the room temperature. The results of the evaluation are presented in Table 4.

The DOE 1 learning was also used to produce fish oil oil-in-water emulsion gummy compositions having increased levels of fish oil (increased relative to the 10 percent by weight used in the previous formulations). Formulations including 20 percent and 34 percent by weight fish oil and the physical properties of samples made according to the respective formulations are shown in Table 5. As indicated in Table 5, as a weight percentage of the oil phase increases, the hardness of the final gummy composition tends to reduce.

TABLE 5
Fish Oil Emulsion Gummy Formulations with
20% and 34% Fish Oil and their Physical Properties

| | | Batch #C1 | Batch #C2 |
|---|---|---|---|
| | Ingredients | wt % | wt % |
| 1 | Water | 19.85% | 15.57% |
| 2 | 250 Bloom Gelatin Porcine | 6.93% | 5.44% |
| | (Gelita) | | |
| 3 | Maltidex Maltitol Syr 5275 | 21.61% | 16.95% |
| | (Cargill) | | |
| 4 | Sugar (Prinova USA) | 16.41% | 12.87% |
| 5 | Xylitol (Danisco) | 11.54% | 9.05% |
| 6 | Aerosil R972 Pharma | 0.12% | 0.22% |
| | (Evonic) | | |
| 7 | Fish Oil TG1055 (Golden | 20.00% | 33.96% |
| | Omega) | | |
| 8 | Masking flavor QN94 | 1.00% | 1.70% |
| | (Virginia Dare) | | |
| 9 | Lemon flavor (Virginia | 2.42% | 4.12% |
| | Dare) | | |
| 10 | Citric acid solution 50% | 0.12% | 0.12% |
| | Total: | 100.00% | 100.00% |
| | Removal time (minutes) | 20 | 15 |
| | Brix (%) | 81.0 | 78.0 |
| | Water Activity | 0.6515 | 0.6985 |
| | TA-8A Hardness (g * Force) | 268 | 226 |

Both fish oils of the triglyceride (TG) form and fish oils of the ethyl ester (EE) form can be used to make a fish oil emulsion gummy composition. Table 6 shows representative formulations of fish oil in water emulsion gummy compositions containing approximately 25 percent fish oil (triglyceride or ethyl ester) by weight. Table 6 also shows the physical properties of sample compositions made according to the representative formulations measured two days after removal from a respective mold.

TABLE 6
Formulations with TG or EE Type of Fish Oil and
their Physical Properties

| | | Batch #D1 | Batch #D2 |
|---|---|---|---|
| | Ingredients | wt % | wt % |
| 1 | Water | 19.63% | 19.14% |
| 2 | 175 Bloom Gelatin Porcine | 6.07% | 5.92% |
| | (Gelita) | | |
| 3 | Xylitol (Danisco) | 27.30% | 26.63% |
| 4 | Maltitol syrup 5275 (Cargill) | 19.83% | 19.33% |
| 5a | Fish oil TG1055 Golden | 23.91% | |
| | Omega | | |
| 5b | Fish oil 150/500 EE | | 25.64% |
| 6 | Masking flavor QN94 | 0.25% | 0.27% |
| | (Virginia Dare) | | |
| 7 | Strawberry flavor (Gold | 0.76% | 0.80% |
| | Coast) | | |
| 8 | Lemon Lime flavor | 0.80% | 0.80% |
| | (Virginiar Dare) | | |
| 9 | Aerosil R972 Pharma | 0.25% | 0.27% |
| | (Evonic) | | |
| 10 | Citric acid solution 50% | 1.20% | 1.20% |
| | Total: | 100.00% | 100.00% |
| | pH | 3.55 | 3.56 |
| | Brix (%) | 75.5 | 74.1 |
| | Water Activity | 0.7332 | 0.7225 |
| | TA-8A Hardness (g * Force) | 159 | 180 |
| | Appearance after 7 days in | shape not | shape not |
| | 40° C./75%RH chamber | changed | changed |

The DOE 1 learning provided insights for fish oil emulsion gummy formulations with structurants other than gelatin, such as pectin, agar-agar, and/or locust bean gum. Representative formulations of a fish oil oil-in-water emulsion gummy composition containing agar-agar, locust bean gum, with and without gelatin, and physical properties of samples made according to such formulations is shown in Table 7.

TABLE 7
Fish Oil Formulation with Different Structurants and
their Physical Properties

| | | Batch | Batch |
|---|---|---|---|
| | | #E1 | #E2 |
| | Ingredients | wt % | wt % |
| 1 | Water | 13.99% | 13.04% |
| 2 | Maltitol syrup 5275 (Cargill) | 31.51% | 30.70% |
| 3 | Sugar (Prinova USA) | 35.00% | 33.42% |
| 4 | Agar-Agar (TIC gum) | 1.50% | 1.63% |
| 5 | Locust Bean Gum (TIC gum) | 0.30% | 0.25% |
| 6 | 250 Bloom Gelatin Porcine | | 3.26% |
| | (Gelita) | | |
| 7 | Fish oil 40/230 VivoMega | 14.85% | 14.85% |
| | 0525 TG60 | | |
| 8 | Masking flavor QN94 | 0.75% | 0.75% |
| | (Virginia Dare) | | |
| 9a | Lemon flavor (Gold Coast) | 2.00% | |
| 9b | Lemon flavor (Virginia Dare) | | 2.00% |
| 10 | Aerosil R972 Pharma (Evonic) | 0.10% | 0.10% |
| | Total: | 100.00% | 100.00% |
| | pH | 5.4 | n/a |
| | Brix (%) | 77.5 | 79.0 |
| | Water Activity | 0.6949 | 0.6556 |
| | TA-8A Hardness(g * Force) | 493 | 420 |

The gummy compositions utilizing the formulations presented in Table 7 were prepared as follows. The structurant agar-agar and locust bean gum were blended with part of the sugar until uniform. The maltitol syrup was separately added to the water and mixed with a Caframo overhead mixer. The blend of structurants and sugar was then added into the water and maltitol syrup mixture, while mixing. The temperature was then increased to 95° C. for 10 minutes. For Batch #E1, the rest of the sugar was added while mixing and the mixing continued for 15 minutes at 95° C. The temperature was then lowered to 80° C. For Batch #E2, gelatin was added and mixed for 10 minutes at 95° C. to hydrate. The rest of the procedure is the same as Batch #E1. For the oil phase, a Silverson L5M-A mixer was used to mix the fish oil and Aerosil R972 and then the masking flavor and lemon flavor were added while mixing until the oil mixture was uniformly distributed. To combine the oil phase with water phase, the oil phase was added into the water phase at 80° C. and homogenized with the Silverson L5M-A at a mixing speed of about 6000 rpm for five minutes. The final samples were then deposited into silicone molds. The physical properties were measured 24 hours after removing the gummy composition from the mold.

Studies have shown that fish oils, particularly fish oils high in polyunsaturated fatty acids, may be prone to oxidation. It has been found that the addition of an antioxidant such as Vitamin E (d-alpha tocopherol) to an oil-in-water emulsion gummy composition including fish oil can inhibit oxidation of the fish oil present. Table 8 presents representative emulsion gummy composition formulations that includes fish oil and Vitamin E and also presents physical properties of samples made according to the formulations.

TABLE 8
Fish Oil Formulation with Vitamin E and its Physical Properties

| | | Batch #F1 | Batch #F2 | Batch #F3 |
|---|---|---|---|---|
| | Ingredients | Wt % | Wt % | Wt % |
| 1 | Water | 18.13% | 11.47% | 16.02% |
| 2 | 270 Bloom Gelatin Porcine | 6.54% | 6.00% | 8.50% |
| | (Gelita) | | | |
| 3 | Maltidex Maltitol Syr 5275 | 24.54% | 15.26% | 30.45% |
| | (Cargill) | | | |
| 4 | Sugar (Prinova USA) | 19.63% | 12.46% | 17.64% |
| 5 | Xylitol (Danisco) | 13.49% | 6.20% | 8.78% |
| 6 | Vitamin E (d-alpha tocopherol) | 0.47% | 0.45% | 0.47% |
| 7 | Fish Oil TG1055 (Golden Omega) | 15.32% | 45.05% | 15.33% |
| 8 | Masking flavor QN94 (Virginia | 0.51% | 0.80% | 0.60% |
| | Dare) | | | |
| 9 | Lemon flavor (Virginia Dare) | 0.50% | 1.45% | 1.35% |
| 10 | Citric acid solution 50% | 0.80% | 0.80% | 0.80% |
| 11 | Yellow color (CHR Hansen) | 0.06% | 0.06% | 0.06% |
| | Total: | 100.00% | 100.00% | 100.00% |
| | Removal time (minutes) | 30 | 19 | 19 |
| | Brix (%) | 81.0 | 82.5 | 83 |
| | Water Activity | 0.7006 | 0.6998 | 0.6856 |
| | TA-8 Hardness (g*Force) | 166 | 264 | 230 |
| | TA-25 Hardness (g*Force) | 623 | 1226 | 883 |
| | TA-25 Chewiness (g*Force) | 565 | 1189 | 697 |

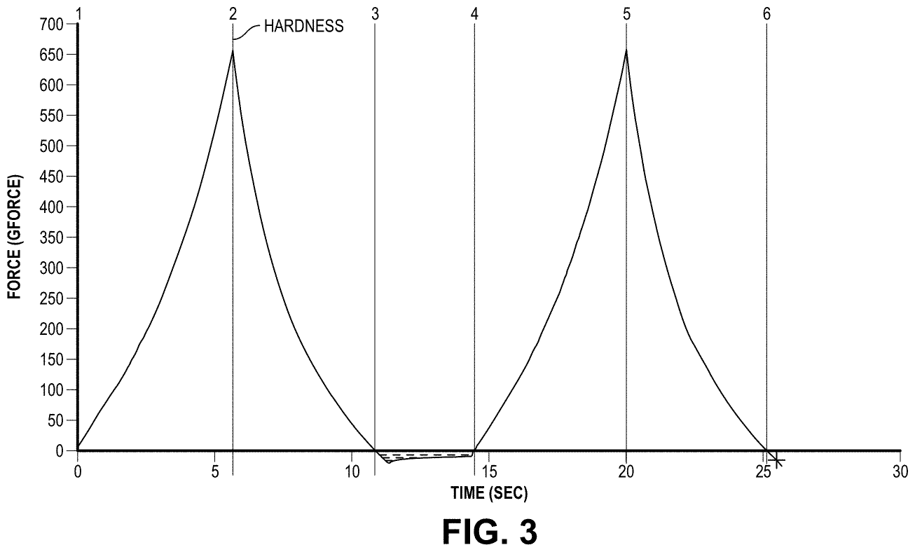

Table 8 shows that the gummy composition samples have properties of ° Brix range within the 77% to 83% range; a water activity less than 0.73; a removal time of 30 minutes or less; and a hardness greater than 150 gForce measured as the peak force to push a TA-8A probe through the sample (a square shape of 2.0 centimeters by 2.0 centimeters with thickness of 0.9 centimeters) from the square top surface for 5 millimeters (mm) at the speed of 0.5 mm/sec. Batch #F2 was made with 45.05 weight percent fish oil demonstrating that high levels of fish oil can be present in an oil-in-water emulsion gummy composition and the gummy composition can meet the target physical property parameters to produce a product that has a relatively rapid removal time, good texture, good heat stability and a pleasant taste. Table 8 also presents hardness measurements using a TA-25 cylindrical probe (diameter 2 inches and height 20 mm). The test was conducted by compressing from a surface of a sample with the probe at 0.5 mm per second to a distance of 70 percent of an original height of the sample, then returning to the original position at the same speed, followed by a one second resting then repeating the compressing and returning with the same conditions. FIG. 3 presents a graph of compression force versus time for the two compressions for Batch #F1. A peak force at the first peak was measured as the hardness. A cohesiveness, springiness and chewiness of a gummy composition may also be calculated based on the two compressions of the TA-25 probe. A cohesiveness is calculated as the area of the second peak divided by the first peak (FIG. 3); the springiness is calculated as the compression distance of the second peak divided by the compression distance of the first peak (FIG. 3); and the chewiness is calculated as the product of the hardness, cohesiveness and springiness (hardness x cohesiveness x springiness). For a fish oil oil-in-water emulsion gummy composition, a target hardness using a TA-25 according to the above-described test is 300 gForce to 1500 gForce, such as 300 gForce to 1300 gForce, such as 300 gForce to 1000 gForce and such as 400 gForce to 900 gForce. A target chewiness is 300 to 1500 such as 300 to 1200, such as 300 to 900 and such as 400 to 800. For a fish oil oil-in-water emulsion, a target hardness using a TA-8A probe to push through the sample (a square shape of 2.0 centimeters (cm) by 2.0 cm with thickness of 0.9 cm) from a top surface for 5 mm at a speed of 0.5 mm/sec. is 150 gForce to 600 gForce. A target removal time is 10 minutes to 30 minutes. The fish oil oil-in-water emulsion gummy composition may be stable at 40° C. for at least seven days, have good texture, good firmness and good heat stability.

II. Mineral Emulsion Gummy Compositions

Certain minerals such as magnesium, calcium and other minerals are important supplements for supporting the human body. Minerals offer support, for example, of bone, nerve, muscle and heart function. Often a recommended daily dosage of a particular mineral or minerals may be difficult to administer in one or more tablets, capsules or gummy composition. The mineral magnesium, for example, has a recommended daily dosage of 420 milligrams (mg) and current gummy composition products generally can contain on the order of 80 mg to 90 mg for a 3 gram (g) to 5 g weight composition. This requires consumption on a daily basis of five to six gummy compositions to meet the recommended daily dosage of magnesium.

It has been found that an oil-in-water emulsion gummy composition can incorporate high amounts of a mineral or minerals into a gummy or gum composition by suspending the mineral(s) into the oil phase first and then dispersing it(them) into water phase uniformly. High mineral oil-in-water emulsion gummy compositions (e.g., 20 percent by weight or more mineral(s) content) have been developed through Designs of Experiment (DOE 2 and DOE 3) to achieve good texture, pleasant taste and good heat stability.

An example of a suitable mineral for an oil-in-water emulsion gummy or gum composition is a mineral in a salt form. Examples include citrates, carbonates or sulfates of magnesium, calcium, zinc or iron. The salt form may be in the form of a solid (e.g., a powder) having a representative particle size on the order of less than 120 microns, such as 80 microns or less. Formulations and processes of magnesium, calcium and their combination were developed with different structurants such as gelatin, pectin, agar-agar, locust bean gum and their combinations. An example of an amount of mineral(s) that may be incorporated in an oil-in-water emulsion gummy composition that demonstrates good texture, pleasant taste and good heat stability is amounts of 20 percent to 40 percent by weight, such as 25 percent by weight, 27 percent by weight, 30 percent by weight, and 35 percent by weight.

An oil-in-water emulsion gummy composition incorporating a high amount of mineral(s) content may be formed with a structurant of gelatin, pectin, locust bean gum, agar agar or a combination of structurants. The oil phase of a gummy or gum composition may contain a lecithin, a polyunsaturated fatty acid, e.g. an omega-3 fatty acid, an omega-6 fatty acid, an omega-9 fatty acid or a vegetable oil, such as one or a mixture of fatty acid esters (for example phospholipids, mono-, di- or tri-glycerides, and lower alkyl esters). Such materials may be natural, synthetic or semi-synthetic. The use of plant and marine oils (e.g. oils from plant seeds (e.g., sunflower oil), algae, fish (especially oily fish), micro-organisms and marine invertebrates (e.g., krill) is especially preferred as is the use of docosahexaenoic acid (DHA) and/or eicosapentaenoic acid (EPA) triglycerides or ethyl esters. An oil phase of a gummy or gum composition may also include medium chain triglyceride (MCT) oils such as coconut or palm kernel oil.

A. Magnesium Emulsion Gummy Formulation

Table 9 shows two magnesium oil-in-water emulsion gummy formulation examples: Batch #G1 used gelatin as the structurant and Batch # G2 used pectin as the structurant. For both examples, the ingredients in the water phase are water, structurant (gelatin or pectin), sugar and sugar alcohol (maltitol). The water phase and oil phase were prepared separately. For Batch #G1, the gelatin was hydrated in water first at about 90° C. for 15 minutes with a Caframo overhead mixer and then maltitol and sugar were added while mixing to be dissolved about 90 to 95° C. For Batch #G2, pectin was hydrated in water first at about 90° C. for 15 minutes and then maltitol syrup and sugar were added. The water phase mixture was then heated to about 100° C. to 105° C. to dissolve the sugar completely into a clear solution. To prepare the oil phase, magnesium citrate particles were suspended in a medium chain triglyceride (MCT) oil and lecithin first with a Caframo overhead mixer. To combine the oil phase with the water phase for Batch #G1, a 50 percent citric acid solution was first added into the gelatin-based water phase at about 70° C. and the oil phase was added while mixing with a Silverson L5M-A mixer at a mixing speed of about 5000 rpm to 6000 rpm. For Batch #G2, the 50 percent citric acid solution was added into the water phase first at about 90° C. and the oil phase was added with the Silverson L5M-A mixer at mixing speed about 6000 rpm. The final samples were then deposited into silicone molds for setting.

TABLE 9
Magnesium Emulsion Gummy Formulation Examples and their Physical
Properties

| | | Batch #G1 | Batch #G2 |
|---|---|---|---|
| | Ingredients | wt % | wt % |
| 1 | Water | 13.28% | 9.89% |
| 2 | 250 Bloom Gelatin Porcine (Gelita) | 3.36% | 0.00% |
| 3 | Pectin (Herbstreith & Fox) | 0.00% | 1.50% |
| 4 | Maltidex Maltitol Syr 5275 | 20.57% | 17.75% |
| | (Cargill) | | |
| 5 | Sugar (Prinova USA) | 16.09% | 19.06% |
| 6 | Magnesium citrate | 27.00% | 32.50% |
| 7 | MCT Oil (Stepan) | 17.00% | 16.60% |
| 8 | Lecithin (American Lecithin | 1.00% | 0.90% |
| | Company) | | |
| 9 | Red Color Fragaria 100 (Chr. | 0.10% | 0.00% |
| | Hansen) | | |
| 10 | Citric Acid Solution 50% | 1.60% | 1.80% |
| | Total: | 100.00% | 100.00% |
| | Brix (%) | 80.8 | 80.0 |
| | Water Activity | 0.7357 | 0.6700 |
| | TA-8A Hardness (g*Force) | 161 | 327 |

Table 10 shows a comparison of the above-described magnesium oil-in-water emulsion gummy composition Batch #G2) to current commercially available gummy composition products. The comparison shows that an oil-in-water emulsion gummy composition can increase a magnesium citrate powder weight concentration in the gummy composition in certain cases by at least 50%.

TABLE 10
Magnesium Citrate Powder Concentration in Current Gummy Products vs.
Emulsion Gummy Composition

| Sample | Magnesium citrate wt % |
|---|---|
| Vitafusion Magnesium gummy | <13.7 |
| Nature Made Magnesium gummy | <21.0 |
| Emulsion gummy Batch #G2 | 32.5 |

B. Calcium Emulsion Gummy Formulations

Table 11 lists calcium oil-in-water emulsion gummy composition formulation examples with different structurants and shows physical properties of samples made according to the formulation examples. The lab batch procedures to prepare the samples were similar to the magnesium emulsion gummy described above. Shown in Table 12 is a comparison of the calcium oil-in-water emulsion gummy compositions to current commercially available gummy composition products. Table 12 demonstrates that the emulsion gummy form allows nearly twice the amount of calcium to the product than currently available gummy products.

TABLE 11
Calcium Emulsion Gummy Formation Examples with Different
Structurants and their Physical Properties

| | | Batch | Batch | Batch |
|---|---|---|---|---|
| | | #H2 | #H3 | #H4 |
| | Ingredients | wt % | wt % | wt % |
| 1 | Water | 10.20% | 10.76% | 11.67% |
| 2 | Agar-Agar (TIC Gums) | 0.95% | 0.79% | 1.12% |
| 3 | 250 Bloom Gelatin Porcine | | 1.12% | |
| | (Gelita) | | | |
| 4 | Locust Bean Gum (TIC Gums) | | | 0.10% |
| 5 | Maltitol Syrup 5275 (Cargill) | 18.53% | 22.22% | 22.00% |
| 6 | Sugar (Prinova USA) | 17.57% | 20.10% | 20.10% |
| 7a | TriCalcium Phosphate powder | 21.50% | | |
| | (Calipharm) | | | |
| 7b | TriCalcium Phosphate | | 24.75% | 24.75% |
| | Ultrafine powder (Jost | | | |
| | Chemical) | | | |
| 8 | MCT Oil (Stepan) | 27.00% | 19.94% | 19.94% |
| 9 | Lecithin(American Lecithin | 1.50% | 0.32% | 0.32% |
| | Company) | | | |
| 10 | Cherry Flavor (Fona) | 0.75% | | |
| 11 | Milk Chocolate Flavor (Fona) | 2.00% | | |
| | Total: | 100.00% | 100.00% | 100.00% |
| | Brix (%) | 80.0 | 82.0 | 83.0 |
| | Water Activity | 0.7431 | 0.6426 | 0.6324 |
| | TA-8A Hardness (g*Force) | 225 | 530 | 527 |

TABLE 12
Tricalcium Phosphate Powder Concentration in Current Gummy
Products vs Emulsion Gummy Composition

| | Tricalcium Phosphate |
|---|---|
| Sample | Wt. % |
| Vitafusion Calcium gummy | <15.0 |
| Nature Made Calcium gummy | <13.0 |
| Emulsion gummy | 24.8 |

C. Calcium and Magnesium Gummy Composition Formulations

1) Formulation with Different Structurants

Table 13 shows calcium and magnesium oil-in-water emulsion gummy composition formulation examples with different structurants and the properties of samples made according to the formulation examples. The lab batch procedures were similar to the calcium or magnesium emulsion gummy compositions described above. In these examples, it was discovered that emulsion gummy compositions made with a combination of agar-agar and locust bean gum provided the most firm and pleasant texture.

TABLE 13
Calcium and Magnesium Emulsion Gummy Formation Examples with
Different Structurant (Gelatin, Agar Agar, Locust Bean Gum) And
their Physical Properties

| | | Batch #I1 | Batch #I2 | Batch #I3 |
|---|---|---|---|---|
| | Ingredients | wt % | wt % | wt % |
| 1 | Water | 15.30% | 10.29% | 11.52% |
| 2 | 250 Bloom Gelatin Porcine | 4.05% | | |
| | (Gelita) | | | |
| 3 | Agar-Agar (TIC Gums) | | 1.03% | 0.54% |
| 4 | Locust Bean Gum (TIC Gums) | | | 0.14% |
| 5 | Maltitol Syrup 5275 (Cargill) | 21.40% | 20.34% | 21.60% |
| 6 | Sugar (Prinova USA) | 17.42% | 17.35% | 20.21% |
| 7 | TriCalcium Phosphate | 5.47% | 6.81% | 6.13% |
| | Ultrafine powder | | | |
| | (Jost Chemical) | | | |
| 8 | Magnesium citrate powder | 15.40% | 19.14% | 17.24% |
| | (Jost Chemical) | | | |
| 9 | MCT Oil (Stepan) | 18.40% | 22.60% | 20.32% |
| 10a | Lecithin (American Lecithin | 0.76% | | |
| | Company) | | | |
| 10b | Lecithin (ADM) | | 0.44% | 0.40% |
| 11 | Red Color Fragaria 100 (Chr. | 0.10% | | |
| | Hansen) | | | |
| 12 | Citric Acid Solution 50% | 0.80% | | |
| 13 | Cherry Flavor (Fona) | | 1.00% | |
| 14 | Milk Chocolate Flavor (Fona) | | 1.00% | 1.00% |
| 15 | 100% CACAO Powder | 1.00% | | 0.90% |
| | (Hershey's) | | | |
| | Total: | 100.00% | 100.00% | 100.00% |
| | pH | 4.31 | 5.97 | 6.11 |
| | Brix (%) | 82 | 81.5 | 78.5 |
| | Water Activity | 0.7172 | 0.6549 | 0.7131 |
| | TA-8A Hardness (g*Force) | 222 | 391 | 156 |

2) Agar-Agar & Locust Bean Gum-Based Formulations

A second DOE (DOE 2) was directed at a magnesium and calcium oil-in-water emulsion gummy composition. The water phase is the continuous phase and contains agar-agar & locust bean gum as the structurant, water, sugar (solid), maltitol syrup and chocolate flavor. The oil phase contains MCT oil as carrier, magnesium citrate powder and tricalcium phosphate powder, lecithin and cocoa powder.

DOE 2 was designed to understand how the ingredients in the water phase and the ratio between oil phase to water phase affect whether a gummy composition sets or not and other physical properties. The ingredients that were kept constant in DOE 2 were the structurant (agar-agar 1.0 weight percent, locust bean gum 0.5 weight percent), flavor (cocoa powder 1.25 weight percent (oil please), chocolate flavor 0.4 weight percent (water phase)), minerals (magnesium citrate powder 18.97 weight percent and tricalcium phosphate powder 6.77 weight percent). The other five ingredients in the DOE 2 formulation (water, sugar, sugar alcohol (maltitol), MCT and lecithin) were treated as variables with following weight percent ranges shown in Table 14.

TABLE 14
DOE 2 Formulation

| | Minimum | Maximum |
|---|---|---|
| Mixture DOE | (Wt. %) | (Wt. %) |
| Water | 9.00 | 13.00 |
| Sugar | 10.00 | 25.00 |
| Maltitol syrup | 10.00 | 25.00 |
| MCT Oil | 17.25 | 28.95 |
| Lecithin | 0.30 | 2.00 |

| | |
|---|---|
| Total: | 71.45 |

25 DOE 2 batches were designed and conducted, and gummy removal time, hardness, water activity, ° Brix, pH and heat stability were tested. Error! Reference source not found. 15 shows two example formulations of the DOE batches that can form emulsion gummies and physical properties of samples made according to the formulations.

For DOE 2 Batch #J1 and #J2, a small part of sugar was blended with agar-agar, locust bean gum, water and maltitol syrup were separately mixed together. The blend of sugar and structurant was then slowly added into the water and maltitol syrup with Caframo overhead mixer. The batch temperature was slowly increased to about 90° C. while mixing and then the structurants were hydrated for 15 minutes. The rest of the sugar was added slowly until it was dissolved completely. To suspend the magnesium citrate and tricalcium phosphate powder into the oil phase at room temperature, MCT oil and lecithin were initially mixed with Caframo overhead mixer at speed about 200 rpm. Magnesium citrate powder and tricalcium phosphate powder were the added with increasing mixing speed to 600 rpm. Finally, the cocoa powder was added. To combine the oil phase with the water phase, the chocolate flavor was added into the water phase first at 70° C. with Silverson L5M-A high shear mixer at 6000 rpm, then the oil phase was added and mixed at about 7000 rpm for about 3 minutes to 5 minutes. The samples were then deposited into silicone molds for setting.

Soon after the gummy compositions were deposited into silicone molds, the removal time was established by pushing gummy composition samples out from the back of a silicone mold and confirming that all sides of the samples were separated from the mold. After the gummy composition samples were removed from the mold they were left to sit in room air for 24 hours. Following the sitting period, the following physical property tests were performed: ° Brix (Brix Refractometer), water activity (Aqualab 4TE water activity meter), hardness (Texture Analyzer from Stable Micro Systems). For the hardness measurement, a TA-8A probe was used to push through the sample from its top surface for 5 mm at speed 0.5 mm/sec. The peak force was measured as the hardness. Five samples were tested, and the average hardness was used. For heat stability study, the samples are packed into bottles and put into a 40° C./75 percent relative humidity (RH %) stability chamber. The sample appearance after seven days was evaluated following removal from the chamber and a return to the room temperature.

TABLE 15
Calcium and Magnesium Emulsion Gummy DOE 2 Batches and
their Physical Properties

| | | Batch #J1 | Batch #J2 |
|---|---|---|---|
| | Ingredients | wt % | wt % |
| 1 | Water | 10.80% | 12.22% |
| 2 | Agar-Agar (TIC Gums) | 1.00% | 1.00% |
| 3 | Locust Bean Gum (TIC Gums) | 0.15% | 0.15% |
| 4 | Sugar (Prinova USA) | 23.24% | 14.13% |
| 5 | Maltitol Syrup 5275 (Cargill) | 17.76% | 25.00% |
| 6 | TriCalcium Phosphate Ultrafine | 6.77% | 6.77% |
| | powder (Jost Chemical) | | |
| 7 | Magnesium citrate powder (Jost | 18.98% | 18.98% |
| | Chemical) | | |
| 8 | MCT Oil (Stepan) | 18.27% | 19.39% |
| 9 | Lecithin (ADM) | 1.37% | 0.72% |
| 10 | 100% CACAO Powder (Hershey's) | 1.25% | 1.25% |
| 11 | Chocolate Flavor (Virginia Dare) | 0.40% | 0.40% |
| | Total: | 100.00% | 100.00% |
| | Removal time (minute) | 10 | 10 |
| | Brix (%) | 80 | 77.5 |
| | Water Activity | 0.6647 | 0.7278 |
| | TA-8A Hardness(g*Force) | 146 | 209 |
| | Appearance after 7 days in | Shape | Shape not |
| | 40° C./75% RH chamber | slightly | changed |
| | | deformed | |

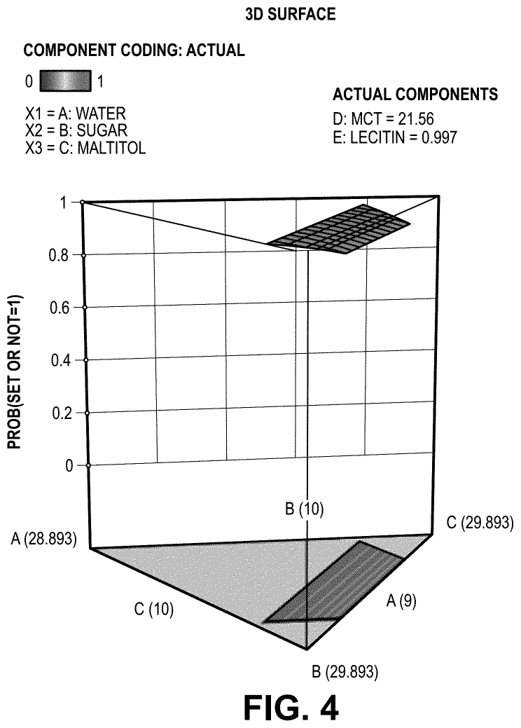

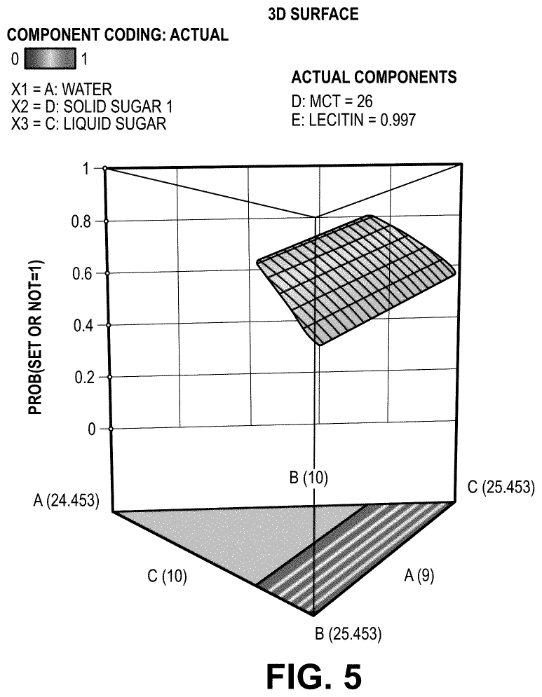

Whether the magnesium and calcium oil-in-water emulsion gummy composition set or not depends on each of the variable ingredients. Water, sugar and maltitol syrup were determined to have a positive impact on the setting while MCT oil and lecithin were determined to have a negative impact on the setting with lecithin having the most negative impact. FIG. 4 and FIG. 5 demonstrate a probability of a gummy composition setting where water, sugar and maltitol syrup amounts in a composition are changed and MCT oil and lecithin are held constant. In FIG. 4, an amount of MCT oil is 21.56 weight percent and in FIG. 5, MCT oil is 26 weight percent. Lecithin is 0.997 weight percent in both FIG. 4 and FIG. 5. A probability of 1 was indicates that a gummy composition will set. FIG. 4 and FIG. 5 representatively show that increasing a weight percent of MCT from 21.56 weight percent to 26 weight percent, greatly reduces the probability of a gummy composition setting.

The variable ingredients in the formulation of DOE 2 were determined to affect the gummy hardness linearly. A water amount in the formulation was determined to increase the hardness the most, followed by MCT, maltitol syrup and sugar. Increasing lecithin in the formula was determined to reduce the hardness.

The DOE 2 modeling was conducted to form magnesium and calcium oil-in-water emulsion gummy composition target formulations based on the targeted criteria: removal time between 10 to 30 minutes, hardness equal or above 150 gForce (when measured using a TA-8A probe to push through the sample from its top surface for 5 mm at the speed of 0.5 mm/sec.), water activity below 0.7300 and a gummy composition shape that did not change after seven days in a 40° C./75% RH chamber. Two confirmation batches were conducted to test the DOE 2 modeling. The formulations and the physical properties of samples made according to the formulations are set forth in Table 16.

TABLE 16
DOE 2 Ca/Mg Formulations and their Physical Properties

| | | Batch #K1 | Batch #K2 |
|---|---|---|---|
| | Ingredients | wt % | wt % |
| 1 | Water | 12.13% | 12.01% |
| 2 | Agar-Agar (TIC Gums) | 1.00% | 1.00% |
| 3 | Locust Bean Gum (TIC Gums) | 0.15% | 0.15% |
| 4 | Sugar (Prinova USA) | 20.04% | 20.90% |
| 5 | Maltitol Syrup 5275 (Cargill) | 17.26% | 17.87% |
| 6 | TriCalcium Phosphate Ultrafine | 6.77% | 6.77% |
| | powder (Jost Chemical) | | |
| 7 | Magnesium citrate powder (Jost | 18.98% | 18.98% |
| | Chemical) | | |
| 8 | MCT Oil (Stepan) | 21.43% | 20.35% |
| 9 | Lecithin (ADM) | 0.59% | 0.32% |
| 10 | 100% CACAO Powder | 1.25% | 1.25% |
| | (Hershey's) | | |
| 11 | Chocolate Flavor (Virginia Dare) | 0.40% | 0.40% |
| | Total: | 100.00% | 100.00% |
| | Set time (minute) | 10 | 15 |
| | Brix (%) | 79.5 | 78.5 |
| | Water Activity | 0.7091 | 0.6973 |
| | TA-8A Hardness (g*Force) | 212 | 213 |
| | Appearance after 7 days in | Shape not | Shape not |
| | 40° C./75% RH chamber | changed | changed |

The emulsion gummy compositions represented by Batch #K1 and K2 were found to have firm, clean first bite texture and leave only small amount of gritty or chalky mouth feel despite the presence of a relatively large amount of calcium and magnesium.

A third Design of Experiment (DOE 3) was designed in an effort to modify the chewiness of a calcium/magnesium emulsion gummy composition. Based on the DOE 2 learning, the MCT oil amount (20.55 weight percent) and lecithin amount (0.45 weight percent) were fixed for the oil phase. The sugar amount (20.5 weight percent) and maltitol amount (17.8 weight percent) were fixed for the water phase. In DOE 3, a structurant of gelatin was added in combination with agar-agar and locust bean gum. The variables were water, gelatin, agar-agar and locust bean gum with the following weight percent ranges shown in Table 17.

TABLE 17
DOE 3 Formulation Variables

| | Minimum | Maximum |
|---|---|---|
| Mixture DOE | (Wt. %) | (Wt. %) |
| Water | 9.0 | 10.3 |
| Gelatin | 2.0 | 3.3 |
| Agar-agar | 0.5 | 1.5 |
| Locust bean gum | 0.1 | 0.55 |

| | |
|---|---|
| Total: | 12.9 |

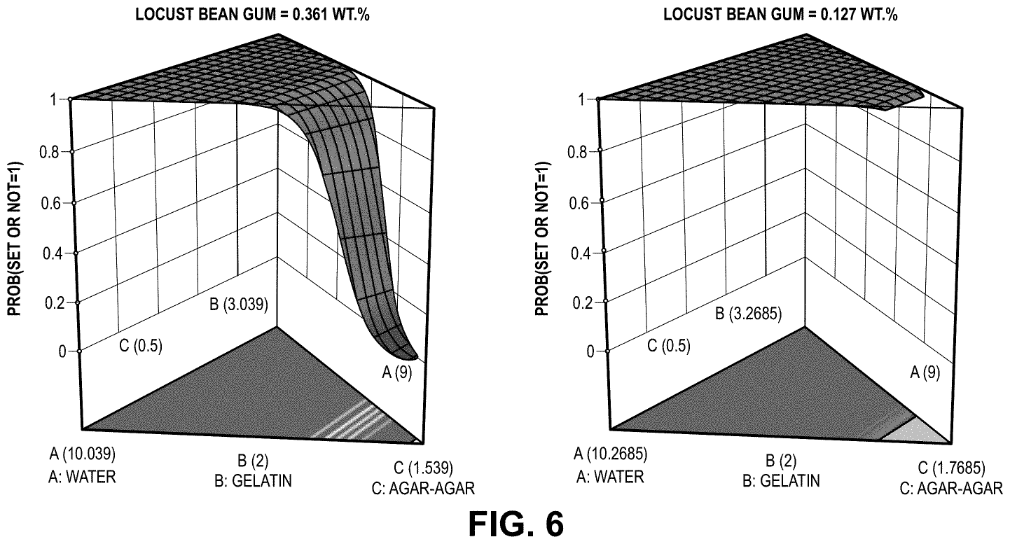

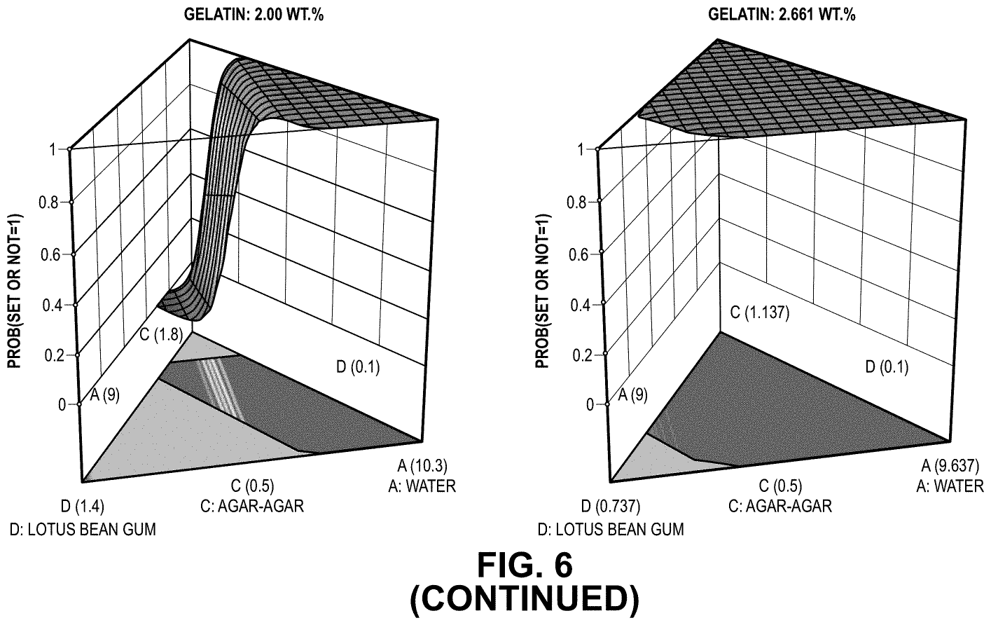

In DOE 3, it was determined that whether a gummy composition sets or not depends on the amount of water and the amounts of each of the three structurants (gelatin, agar-agar and locust bean gum). FIG. 6 shows three-dimensional plots of probability of setting of a gummy composition from 0 to 1 with 1 being the greatest probability of setting. In 1) of FIG. 6, an amount of locust bean gum is held constant at 0.361 weight percent and water, gelatin and agar-agar amounts are varied. In 2) of FIG. 6, an amount of locust bean gum is held constant at 0.127 weight percent and water, gelatin and agar-agar amounts are varied. In 3) of FIG. 6, an amount of gelatin is held constant at 2.00 weight percent and water, locust bean gum and agar-agar amounts are varied. In 4) of FIG. 6, an amount of gelatin is held constant at 2.661 weight percent and water, locust bean gum and agar-agar amounts are varied. FIG. 6 shows that amounts of gelatin and water have positive effects on setting whereas amounts of locust bean gum and agar-agar tend to have negative effects.

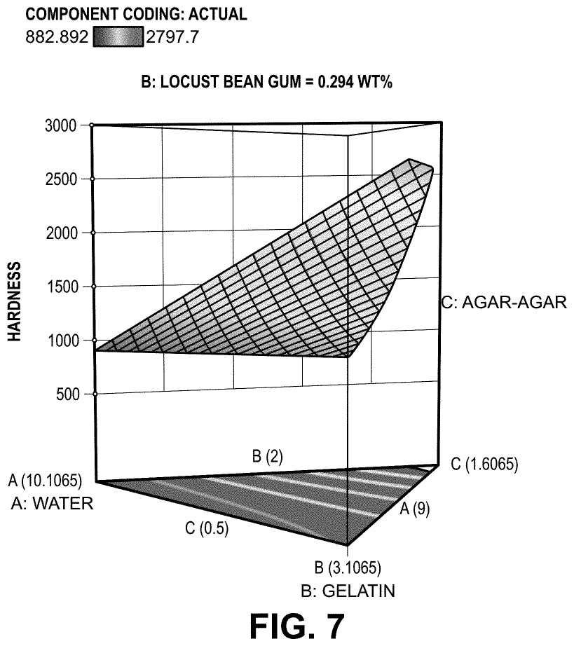

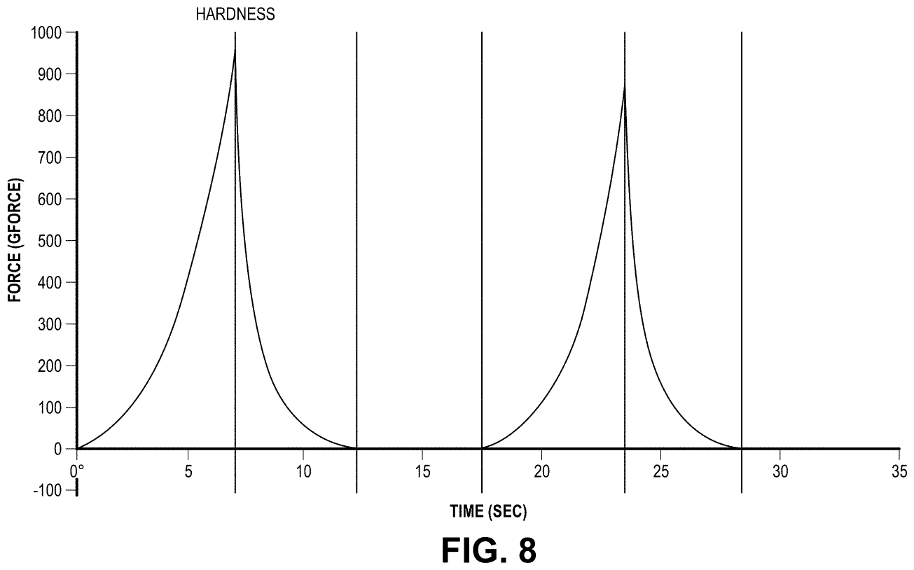

DOE 3 also shows that the hardness of a gummy composition depends on the amount of water and the amounts of each of the three structurants (gelatin, agar-agar and locust bean gum). FIG. 7 shows a three-dimensional plot of hardness with a locust bean gum amount maintained at 0.294 weight percent and an amount of water, agar-agar and gelatin varied. For the hardness analysis, a double compression test was performed using a TA-25 cylindrical probe (diameter 2 inches and height 20 mm). The test was conducted by compressing from a surface of a sample with the probe at 0.5 mm per second to a distance of 70 percent of an original height of the sample, then returning to the original position at the same speed, followed by a one second resting then repeating the compressing and returning with the same conditions. FIG. 8 shows a plot of the hardness versus time for the two compressions. A peak force at the first peak was measured as the hardness. It was determined that that an amount of agar-agar has the greatest effect on hardness.

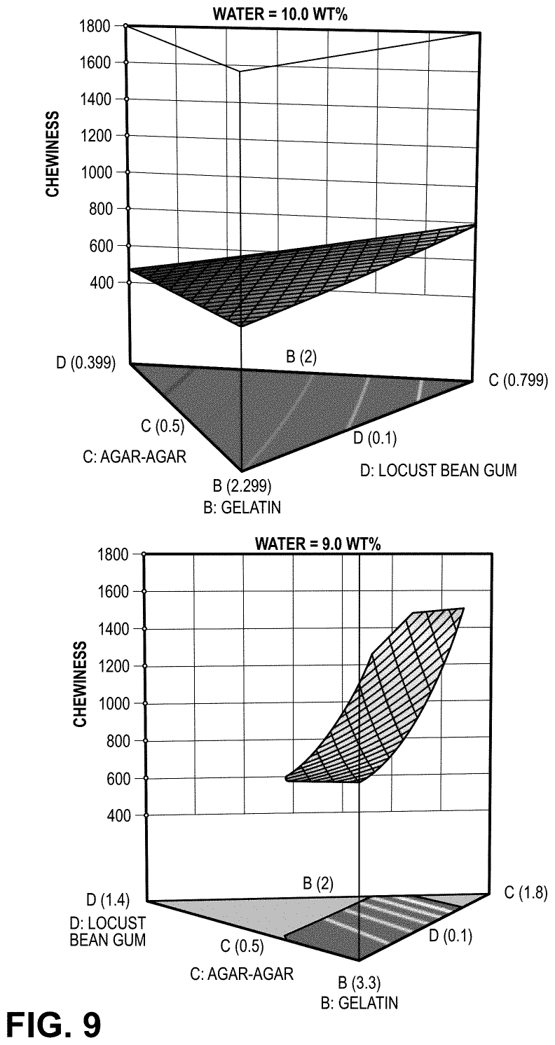

In DOE 3, chewiness of a gummy composition was also studied. For the chewiness analysis, the compressions of the TA-25 probe were used to determine a cohesiveness (the area of the second compression peak divided by the first peak) and a springiness (the compression distance of the second peak divided by the first peak). A chewiness was then calculated as a product of the hardness, the cohesiveness and the springiness (hardness×cohesiveness×springiness). Chewiness was shown to also depend on the amount of water and the amounts of each of the three structurants (gelatin, agar-agar and locust bean gum). FIG. 9 shows two, three-dimensional plots of chewiness with an amount of water held constant in a composition and an amount of each structurant changed. In 1) of FIG. 9, an amount of water was 10.0 percent by weight and in 2) of FIG. 9, an amount of water was 9.0 percent by weight. The plots in FIG. 9 show that when water is 10 percent by weight, a chewiness of a gummy composition did not exceed 800 gForce while when an amount of water was reduced to 9.0 percent by weight, a chewiness could vary between 600 gForce and 1500 gForce depending on the amounts of the three structurants.

Table 18 shows representative examples of emulsion gummy formulations including calcium and magnesium with combined structurants of agar-agar, locust bean gum and gelatin. The physical properties of samples made according to the emulsion gummy formulations are also presented.

TABLE 18
Calcium and Magnesium Emulsion Gummy Formulation Examples with
Gelatin, Agar-agar & Locust Bean Gum and their Physical Properties

| | | Batch #L1 | Batch #L2 |
|---|---|---|---|
| | Ingredients | wt % | wt % |
| 1 | Water | 9.30% | 9.35% |
| 2 | Agar-Agar (TIC Gums) | 1.50% | 0.84% |
| 3 | Locust Bean Gum (TIC Gums) | 0.10% | 0.39% |
| 4 | 250 Bloom Gelatin Porcine (Gelita) | 2.00% | 2.32% |
| 5 | Sugar (Prinova USA) | 20.50% | 20.50% |
| 6 | Maltitol Syrup 5275 (Cargill) | 17.80% | 17.80% |
| 7 | TriCalcium Phosphate Ultrafine | 6.69% | 6.69% |
| | powder (Jost Chemical) | | |
| 8 | Magnesium citrate powder (Jost | 18.86% | 18.86% |
| | Chemical) | | |
| 9 | MCT Oil (Stepan) | 20.55% | 20.55% |
| 10 | Lecithin (ADM) | 0.45% | 0.45% |
| 11 | 100% CACAO Powder (Hershey's) | 1.25% | 1.25% |
| 12 | Chocolate Flavor (Fona) | 1.00% | 1.00% |
| | Total: | 100.00% | 100.00% |
| | Removal time (minutes) | 10 | 10 |
| | Brix (%) | 76.5 | 78 |
| | Water Activity | 0.7344 | 0.7381 |
| | TA-8A Hardness (g*Force) | 393 | 350 |
| | TA-25 Hardness (g*Force) | 2798 | 1383 |
| | TA-25 Chewiness (g*Force) | 1667 | 850 |
| | Appearance after 7 days in | Shape | Shape not |
| | 40° C./75% RH chamber | slightly | changed |
| | | deformed | |

To modify the texture of an emulsion gummy composition samples containing powdered ingredients such as minerals (e.g., calcium, magnesium, etc.), glycerin was added to the composition. Adding an amount of glycerin was also shown to reduce the water activity of a gummy composition. Table 19 shows a representative example of an emulsion gummy formulation including calcium and magnesium with combined structurants of agar-agar, locust bean gum and gelatin. The physical properties of a sample made according to the emulsion gummy formulation is also presented.

TABLE 19
Calcium and Magnesium Emulsion Gummy Formulation Example
with Gelatin, Agar-Agar & Locust Bean Gum Structurants and Glycerin
and its Physical Properties

| | | Batch #M1 |
|---|---|---|
| | Ingredients | wt % |
| 1 | Water | 8.58% |
| 2 | Agar-Agar (TIC Gums) | 0.56% |
| 3 | Locust Bean Gum (TIC Gums) | 0.28% |
| 4 | 250 Bloom Gelatin Porcine (Gelita) | 2.72% |
| 5 | Sugar (Prinova USA) | 18.03% |
| 6 | Maltitol Syrup 5275 (Cargill) | 18.03% |
| 7 | Glycerin | 3.00% |
| 8 | TriCalcium Phosphate Ultrafine | 6.69% |
| | powder (Jost Chemical) | |
| 9 | Magnesium citrate powder (Jost | 18.86% |
| | Chemical) | |
| 10 | MCT Oil (Stepan) | 20.55% |
| 11 | Lecithin (ADM) | 0.45% |
| 12 | 100% CACAO Powder (Hershey's) | 1.25% |
| 13 | Chocolate Flavor (Fona) | 1.00% |
| | Total: | 100.00% |
| | Removal time (minutes) | 30 |
| | Brix (%) | 80.5 |
| | Water Activity | 0.6547 |
| | TA-8A Hardness (g*Force) | 274 |
| | TA-25 Hardness (g*Force) | 1043 |
| | TA-25 Chewiness (g*Force) | 739 |

Herbal or botanical dietary supplements have been used to promote health and wellness for years. It has been found that an oil-in-water emulsion gummy composition can incorporate high amounts of an herb or herbs into a gummy or gum composition by suspending the herb(s) into the oil phase first and then dispersing it(them) into water phase uniformly. The results of the DOE 2 and DOE 3 modeling proved effective for herbal or botanical gummy composition formulation. Table 20 shows a gummy composition formulation including turmeric extract as an active and shows the resulting properties of a gummy composition sample formed according to the formulation. Glycerin was also included as the turmeric extract used was a powder.

TABLE 20
Emulsion Gummy Composition Including Herbal Active and its Physical
Properties

| | | Batch #N1 |
|---|---|---|
| | Ingredients | Wt % |
| 1 | Water | 8.80% |
| 2 | 250 Bloom Gelatin Porcine | 4.40% |
| 3 | Maltitol | 15.95% |
| 4 | Sugar | 20.35% |
| 5 | Xylitol | 5.50% |
| 7 | Glycerin | 3.00% |
| 8 | MCT Oil (Stepan) | 22.74% |
| 9 | Tumeric Extract 95% | 18.06% |
| | (OmniActive) | |
| 10 | Lecithin (ADM) | 0.40% |
| 11 | Pineapple orange Flavor (Fona) | 0.80% |
| | Total: | 100.00% |
| | Removal time (minutes) | 30 |
| | Brix (%) | 81.5 |
| | Water Activity | 0.6173 |
| | TA-8A Hardness (g*Force) | 215 |
| | TA-25 Hardness (g*Force) | 990 |
| | TA-25 Chewiness (g*Force) | 835 |

Table 21 is a comparison of a curcumin (turmeric extract) oil-in-water emulsion gummy composition to current commercially available gummy composition products. Table 21 demonstrates that the emulsion gummy form allows more than twice the amount of curcumin to the product than currently available gummy products.

TABLE 21
Curcumin Powder Concentration in Current Gummy Products vs Emulsion
Gummy Composition

| Sample | Curcumin Wt % |
|---|---|
| Vitafusion Turmeric Curcumin gummy | <8.5 |
| MaryRuth's Turmeric gummy | <3.3 |
| Emulsion gummy | 18.0 |

Target physical properties for an oil-in-water emulsion gummy composition including amounts of mineral(s) and/or herbs of 15 percent by weight or more include a ° Brix of 77 to 83, such as 78 to 81 and a water activity of less than 0.73, such as less than 0.70. A target hardness a target hardness using a TA-8A probe to push through the sample (a square shape of 2.0 centimeters (cm) by 2.0 cm with thickness of 0.9 cm) from a top surface for 5 mm at a speed of 0.5 mm/sec. is 150 gForce to 600 gForce. A target hardness measured as a peak force of two separate compressions from a surface of a gummy composition sample with a TA-25 cylindrical probe at 0.5 mm per second to a distance of 70 percent of an original height of the sample is 800 gForce to 3000 gForce, such as 1000 gForce to 2500 gForce. A target chewiness calculated as a product of the hardness, the cohesiveness and the springiness (hardness×cohesiveness×springiness) as described above is 600 gForce to 900 gForce, such as 650 gForce to 900 gForce. A target removal time is 10 minutes to 30 minutes. The oil-in-water emulsion gummy composition including amounts of mineral(s) and/or herbs may be stable at 40° C. for at least seven days, have good texture, good firmness and good heat stability.

III. Mineral Emulsion Gummy Including Fish Oil

In Section I, oil-in-water emulsion gummy compositions were formed using fish oil as the active ingredient (dietary or nutritional active) and a source of the oil. In section II, oil-in-water emulsion gummy compositions were formed using minerals or herbs as the active ingredient. In this section, actives of both fish oil and mineral(s) and/or herb(s) may be combined in an oil-in-water emulsion gummy composition. Table 22 presents an emulsion gummy composition formulation including fish oil and calcium and magnesium and shows physical properties of a sample composition made according to the formulation.

TABLE 22
Calcium and Magnesium and Fish Oil Emulsion Gummy and its Physical
Properties

| | | Batch #01 |
|---|---|---|
| | Ingredients | wt % |
| 1 | Water | 12.13% |
| 2 | Agar-Agar (TIC Gums) | 1.00% |
| 3 | Locust Bean Gum (TIC Gums) | 0.15% |
| 4 | Sugar (Prinova USA) | 20.03% |
| 5 | Maltitol Syrup 5275 (Cargill) | 17.26% |
| 6 | TriCalcium Phosphate Ultrafine powder | 6.77% |
| | (Jost Chemical) | |
| 7 | Magnesium citrate powder (Jost Chemical) | 18.98% |
| 8 | MCT Oil (Stepan) | 16.53% |
| 9 | Fish oil 40/230 VivoMega 0525 TG60 | 4.90% |
| 10 | Lecithin | 0.59% |
| 11 | 100% CACAO Powder (Hershey's) | 1.25% |
| 12 | Chocolate Flavor (Virginia Dare) | 0.40% |
| | Total: | 100.00% |
| | Brix (%) | 81.5 |
| | Water Activity | 0.6778 |
| | TA-8A Hardness (g*Force) | 285 |

### ASPECTS OF THE INVENTION

Aspect 1. A gummy composition comprising a set emulsification of an oil phase in a water phase, wherein the water phase comprises a structurant, and wherein the gummy composition has a hardness of 150 gForce or greater (e.g., a hardness of 150 gForce to 600 gForce), a water activity below 0.73 such as a hardness of 150 gForce to 600 gForce, a water activity below 0.73, such as below 0.70 and a ° Brix of 77 to 83 or a ° Brix of 78 to 81.

Aspect 2. The gummy composition of Aspect 1, wherein the oil phase comprises fish oil.

Aspect 3. The gummy composition of Aspect 2, wherein the fish oil is present in an amount of at least 10 percent by weight of the composition.

Aspect 4. The gummy composition of Aspect 2 or Aspect 3, wherein the fish oil is present in an amount of between 10 percent and 50 percent by weight of the composition, such as 20 percent by weight, 34 percent by weight, and 45 percent by weight.

Aspect 5. The gummy composition of any of Aspects 2-4, wherein the fish oil comprises a fish oil in triglyceride form.

Aspect 6. The gummy composition of any of Aspects 2-5, wherein the fish oil comprises a fish oil in an ester form.

Aspect 7. The gummy composition of any of Aspects 2-6, further comprising an amount of an antioxidant such as Vitamin E.

Aspect 8. The gummy composition of any of Aspects 1-7, wherein the structurant comprises gelatin.

Aspect 9. The gummy composition of any of Aspects 1-8, wherein the structurant comprises at least one of locust bean gum and agar-agar or a combination of gelatin, locust bean gum and agar-agar.

Aspect 10. The gummy composition of any of Aspects 1-9, wherein the oil phase comprises a mineral salt or herb, such as a mineral salt of herb, such as a mineral salt or herb in an amount of at least 15 percent by weight of the composition, such as at least 18 percent by weight or such as 20 percent by weight or more.

Aspect 11. The gummy composition of Aspect 10, wherein the oil phase comprises lecithin.

Aspect 12. The gummy composition of Aspect 10 or Aspect 11, wherein the mineral salt comprises an amount of each of a calcium salt and a magnesium salt.

Aspect 13. The gummy composition of any of Aspects 10-12, further comprising an amount of glycerin.

Aspect 14. A method comprising:

administering a gummy composition comprising a set emulsification of an oil phase in a water phase and the gummy composition comprises a hardness of 150 gForce or greater and a water activity below 0.73 such as a hardness of 150 gForce to 600 gForce, a water activity below 0.73, such as below 0.70 and a ° Brix of 77 to 83.

Aspect 15. The method of Aspect 14, wherein the oil phase comprises fish oil and the fish oil is present in an amount of at least 10 percent by weight of the composition.

Aspect 16. The method of Aspect 14 or Aspect 15, wherein oil phase comprises fish oil and the fish oil is present in an amount of between 10 percent and 50 percent by weight of the composition, such as 20 percent by weight, 34 percent by weight, 42.5 percent by weight and 45 percent by weight.

Aspect 17. The method of any of Aspects 14-16, wherein the gummy composition comprises Vitamin E.

Aspect 18. The method of any of Aspects 14-17, wherein the structurant comprises gelatin.

Aspect 19. The method of any of Aspects 14-18, wherein the structurant comprises at least one of locust bean gum and agar-agar or a combination of gelatin, locust bean gum and agar-agar.

Aspect 20. The method of any of Aspects 14-19, wherein the gummy composition comprises a mineral salt or an herb, such as a mineral salt or herb in an amount of at least 15 percent by weight of the composition, such as at least 18 percent by weight or such as 20 percent by weight or more.

Aspect 21. The method of any of Aspects 14-20, wherein the gummy composition comprises lecithin.

Aspect 22. The method of any of Aspects 14-21, wherein the gummy composition comprises glycerin.

Aspect 23. A method of manufacture of a gummy composition comprising a hardness of 150 gForce or greater, such as 150gForce to 600 gForce, and a water activity below 0.73, such as less than 0.70, the method comprising:

emulsifying an oil phase in a water phase, wherein the water phase comprises a structurant; and

depositing the emulsification into a mold for a set time of 30 minutes or less.

Aspect 24. The method of Aspect 23, wherein the oil phase comprises fish oil and the fish oil is present in an amount of at least 10 percent by weight of the composition.

Aspect 25. The method of Aspect 23 or Aspect 24, wherein oil phase comprises fish oil and the fish oil is present in an amount of between 10 percent and 50 percent by weight of the composition, such as 20 percent by weight, 34 percent by weight, 42.5 percent by weight and 45 percent by weight.

Aspect 26. The method of any of Aspects 23-25, wherein the structurant comprises gelatin.

Aspect 27. The method of any of Aspects 23-26, wherein the structurant comprises at least one of locust bean gum and agar-agar or a combination of gelatin, locust bean gum and agar-agar.

Aspect 28. The method of any of Aspects 23-27, wherein prior to emulsifying the oil phase in the water phase, the method comprises suspending a mineral salt or herb in the oil phase, such as a mineral salt or herb in an amount of at least 15 percent by weight of the composition, such as at least 18 percent by weight or such as 20 percent by weight or more.

Aspect 29. The method of Aspect 28, wherein the mineral salt comprises at least one of a calcium salt and a magnesium salt.

Aspect 30. The method of any of Aspects 23-28, wherein the oil phase comprises lecithin.

Whereas specific aspects of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of the invention which is to be given the full breadth of the claims and aspects appended and any and all equivalents thereof.

What is claimed is:

1. A gummy composition comprising a set emulsification of an oil phase in a water phase, wherein the water phase comprises a structurant, and wherein the gummy composition has a hardness of 150 gForce or greater and a water activity below 0.73.

2. The gummy composition of claim 1, wherein the oil phase comprises fish oil.

3. The gummy composition of claim 2, wherein the fish oil is present in an amount of at least 10 percent by weight of the composition.

4. The gummy composition of claim 2, wherein the fish oil is present in an amount of between 10 percent and 50 percent by weight of the composition.

5. The gummy composition of claim 2, wherein the fish oil comprises a fish oil in triglyceride form.

6. The gummy composition of claim 2, wherein the fish oil comprises a fish oil in an ester form.

7. The gummy composition of claim 2, further comprising an amount of Vitamin E.

8. The gummy composition of claim 1, wherein the structurant comprises gelatin.

9. The gummy composition of claim 1, wherein the structurant comprises at least one of locust bean gum and agar-agar.

10. The gummy composition of claim 1, wherein the oil phase comprises a mineral salt or herb.

11. The gummy composition of claim 10, wherein the oil phase comprises lecithin.

12. The gummy composition of claim 11, wherein the mineral salt comprises an amount of each of a calcium salt and a magnesium salt.

13. The gummy composition of claim 10, further comprising an amount of glycerin.

14. A method comprising:
administering a gummy composition comprising a set emulsification of an oil phase in a water phase and the gummy composition comprises a hardness of 150 gForce or greater and a water activity below 0.73.

15. The method of claim 14, wherein the oil phase comprises fish oil and the fish oil is present in an amount of at least 10 percent by weight of the composition.

16. The method of claim 14, wherein oil phase comprises fish oil and the fish oil is present in an amount of between 10 percent and 50 percent by weight of the composition.

17. The method of claim 15, wherein the gummy composition comprises Vitamin E.

18. The method of claim 14, wherein the structurant comprises gelatin.

19. The method of claim 14, wherein the structurant comprises at least one of locust bean gum and agar agar.

20. The method of claim 14, wherein the gummy composition comprises a mineral salt or an herb.

21. The method of claim 20, wherein the gummy composition comprises lecithin.

22. The method of claim 20, wherein the gummy composition comprises glycerin.
